(19) United States
(12) Patent Application Publication
(10) Pub. No.: US 2015/0374247 A1
(43) Pub. Date: Dec. 31, 2015

(54) METHOD OF MEASURING BLOOD FLOW VELOCITY PERFORMED BY MEDICAL IMAGING APPARATUS, AND THE MEDICAL IMAGING APPARATUS

(71) Applicants: SAMSUNG ELECTRONICS CO., LTD., Suwon-si (KR); KOREA ADVANCED INSTITUTE OF SCIENCE AND TECHNOLOGY, Daejeon (KR)

(72) Inventors: Hyun-wook PARK, Daejeon (KR); Joon-sung CHOI, Daejeon (KR)

(73) Assignees: SAMSUNG ELECTRONICS CO., LTD., Suwon-si (KR); KOREA ADVANCED INSTITUTE OF SCIENCE AND TECHNOLOGY, Daejeon (KR)

(21) Appl. No.: 14/690,561

(22) Filed: Apr. 20, 2015

Related U.S. Application Data
(60) Provisional application No. 62/018,757, filed on Jun. 30, 2014.

(30) Foreign Application Priority Data
Aug. 22, 2014 (KR) 10-2014-0109963

(51) Int. Cl. A61B 5/026; A61B 5/055

(52) U.S. Cl. CPC A61B 5/0263; A61B 5/055; A61B 2576/00

(57) ABSTRACT

A method of measuring a blood flow velocity of blood flowing in an object includes obtaining first slab data of a first imaging slab to which a first bipolar gradient is applied, obtaining second slab data of a second imaging slab to which a second bipolar gradient is applied, the second imaging slab being moved to a location different from a location of the first imaging slab, and calculating the blood flow velocity based on data included in slices of the first slab data and slices of the second slab data, the slices of the first slab data being located at a same location as the slices of the second slab data on the object.

### CROSS-REFERENCE TO RELATED APPLICATIONS

This application claims the benefit of U.S. Provisional Application No. 62/018,757, filed on Jun. 30, 2014, in the U.S. Patent and Trademark Office, and Korean Patent Application No. 10-2014-0109963, filed on Aug. 22, 2014, in the Korean Intellectual Property Office, the disclosures of which are incorporated herein in their entirety by reference.

### BACKGROUND

1. Field

One or more exemplary embodiments relate to a method of measuring a blood flow velocity performed by a medical imaging apparatus, and the medical imaging apparatus.

2. Description of the Related Art

Various methods are provided to photograph the inside of a human body by using a medical imaging apparatus. For example, X-ray angiography, X-ray computer tomography, or magnetic resonance angiography may be used to photograph blood vessels.

Recently, in order to increase utility of magnetic resonance angiography, it may be required to simultaneously provide an image having high contrast of surrounding tissues, such as blood vessels, and information about blood flow velocity.

### SUMMARY

One or more exemplary embodiments may provide a method of measuring a blood flow velocity and a medical imaging apparatus, wherein a blood flow velocity is measured based on slices at a same location on an object.

Additional aspects will be set forth in part in the description which follows and, in part, will be apparent from the description, or may be learned by practice of the presented exemplary embodiments.

According to an aspect of an exemplary embodiment, there is provided a method of measuring a blood flow velocity of blood flowing in an object, the method including: obtaining first slab data of a first imaging slab to which a first bipolar gradient is applied; obtaining second slab data of a second imaging slab to which a second bipolar gradient is applied, the second imaging slab being moved to a location different from a location of the first imaging slab; and calculating the blood flow velocity based on data included in slices of the first slab data and slices of the second slab data, the slices of the first slab data being located at a same location as the slices of the second slab data on the object.

The first bipolar gradient may be a gradient magnetic field that sequentially has positive (+) and negative (−) gradients, and the second bipolar gradient may be a gradient magnetic field that has gradients having opposite polarities from and same magnitudes as the first bipolar gradient.

The first bipolar gradient and the second bipolar gradient may each be generated in at least one of an x-axis direction, a y-axis direction, and a z-axis direction.

The calculating of the blood flow velocity may include obtaining first slice data and second slice data at a same location on the object, wherein the first slice data may be extracted from the first slab data and the second slice data may be extracted from the second slab data.

The calculating of the blood flow velocity may include calculating the blood flow velocity by using a phase difference between images generated based on the first slice data and the second slice data.

The method may further include generating an image including information about the calculated blood flow velocity, based on the first slice data and the second slice data.

The obtaining of the first slab data and the obtaining of the second slab data may include sampling the first slab data and the second slab data at a sampling rate lower than a reference sampling rate.

A location of the first imaging slab and a location of the second imaging slab may differ from each other by at least one slice unit.

The obtaining of the first slab data and the obtaining of the second slab data may include obtaining the first slab data and the second slab data based on radial sampling.

According to another aspect of an exemplary embodiment, there is provided a medical imaging apparatus including: a signal transceiver configured to obtain first slab data of a first imaging slab to which a first bipolar gradient is applied, and obtain second slab data of a second imaging slab to which a second bipolar gradient is applied, the second imaging slab being moved to a location different from a location of the first imaging slab; and an operating device configured to calculate a blood flow velocity of blood flowing in an object based on data included in slices of the first slab data and the second slab data, the slices of the first slab data being located at a same location as the slices of the second slab data on the object.

The first bipolar gradient may be a gradient magnetic field that sequentially has positive (+) and negative (−) gradients, and the second bipolar gradient may be a gradient magnetic field that has gradients having opposite polarities from and same magnitudes as the first bipolar gradient.

The first bipolar gradient and the second bipolar gradient may each be generated in at least one of an x-axis direction, a y-axis direction, and a z-axis direction.

The signal transceiver may be configured to obtain first slice data and second slice data at a same location on the object, wherein the first slice data may be extracted from the first slab data and the second slice data may be extracted from the second slab data.

The operating device may be configured to calculate the blood flow velocity by using a phase difference between images generated based on the first slice data and the second slice data.

The operating device may be configured to generate an image including information about the calculated blood flow velocity, based on the first slice data and the second slice data.

The signal transceiver may be configured to obtain the first slab data and the second slab data by sampling the first slab data and the second slab data at a sampling rate lower than a reference sampling rate.

A location of the first imaging slab and a location of the second imaging slab may differ from each other by at least one slice unit.

The transceiver may be configured to obtain the first slab data and the second slab data based on radial sampling.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a block diagram of a medical imaging apparatus according to an exemplary embodiment;

FIG. 2 is a flowchart of a method of measuring a blood flow velocity, according to an exemplary embodiment;

FIG. 3 is a diagram for describing magnetic resonance angiography;

FIGS. 4A and 4B are respectively a pulse sequence mimetic diagram and a diagram for describing phases of a static tissue and blood flow being shifted according to a pulse sequence, according to an exemplary embodiment;

FIGS. 5 and 6 are diagrams for describing a method of measuring a blood flow velocity for a medical imaging apparatus, according to an exemplary embodiment;

FIG. 7 is a flowchart of a method of reconstructing an image having high contrast of a blood flow in a blood vessel, the image including blood flow velocity information, for a medical imaging apparatus, according to an exemplary embodiment;

FIG. 8 is a diagram for describing a method of reconstructing an image having high contrast of a blood flow in a blood vessel, the image including blood flow velocity information, for a medical imaging apparatus, according to an exemplary embodiment;

FIG. 9 is a diagram of an example of an image generated by a medical imaging apparatus;

FIG. 10 is a diagram for describing a result of comparing an image reconstructed according to an exemplary embodiment and an image reconstructed via full sampling; and

FIG. 11 is a block diagram of a medical imaging apparatus according to an exemplary embodiment.

### DETAILED DESCRIPTION

One or more exemplary embodiments will now be described more fully with reference to the accompanying drawings. The exemplary embodiments may, however, be embodied in many different forms and should not be construed as being limited to the exemplary embodiments set forth herein; rather, these exemplary embodiments are provided so that this disclosure will be thorough and complete, and will fully convey the concept of the exemplary embodiments to those of ordinary skill in the art.

Terms used herein will now be briefly described and then one or more exemplary embodiments will be described in detail.

General terms used herein which are widely used are selected while considering functions in one or more exemplary embodiments , but the terms used herein may differ according to intentions of one of ordinary skill in the art, precedents, or emergence of new technologies. Also, in some cases, an applicant arbitrarily selects a term, and in this case, the meaning of the term will be described in detail herein. Accordingly, the terms shall be defined based on the meanings and details throughout the specification, rather than the simple names of the terms.

When something “includes” a component, another component may be further included unless specified otherwise. The term “unit” used in the present specification may refer to a software component, or a hardware component such as a field programmable gate array (FPGA) or an application specific integrated circuit (ASIC), and may perform a certain function. However, the “unit” is not limited to software or hardware. The “unit” may be configured in an addressable storage medium and may be configured to be executed by one or more processors. Hence, the “unit” includes elements such as software elements, object-oriented software elements, class elements, and task elements, and processes, functions, attributes, procedures, sub-routines, segments of program codes, drivers, firmware, micro-codes, circuits, data, databases, data structures, tables, arrays, and variables. The functions provided in the elements and the units may be combined into a fewer number of elements and units or may be divided into a larger number of elements and units.

While describing one or more exemplary embodiments, descriptions about drawings that are not related to the one or more exemplary embodiments are omitted. The exemplary embodiments may have different forms and should not be construed as being limited to the descriptions set forth herein. Those components that are the same or are in correspondence are rendered with the same reference numeral regardless of the figure number, and redundant explanations are omitted.

As used herein, the term “and/or” includes any and all combinations of one or more of the associated listed items. Expressions such as “at least one of,” when preceding a list of elements, modify the entire list of elements and do not modify the individual elements of the list.

In the present specification, the term “image” may refer to multi-dimensional data composed of discrete image elements (e.g., pixels in a two-dimensional (2D) image and voxels in a three-dimensional (3D) image). For example, an image may include a medical image of an object acquired by using an X-ray, computed tomography (CT), magnetic resonance imaging (MRI), ultrasonic waves, or another medical image photographing apparatus.

Furthermore, in the present specification, the term “object” may refer to a person or an animal, or a part of a person or an animal. For example, the object may include the liver, the heart, the womb, the brain, a breast, the abdomen, or a blood vessel. Furthermore, the “object” may include a phantom. The phantom may refer to a material having a volume that is approximately the intensity and effective atomic number of a living thing, and may include a sphere phantom having a property similar to a human body.

Furthermore, in the present specification, the term “user” may refer to a medical professional, such as a doctor, a nurse, a medical laboratory technologist, and an engineer who repairs a medical apparatus, but the user is not limited thereto.

Furthermore, in the present specification, the term “medical imaging apparatus” may refer to an apparatus using an X-ray, CT, MRI, or ultrasonic waves, or may be another medical image system, but for convenience of description herein, the term “medical imaging apparatus” may refer to an MRI apparatus.

Furthermore, in the present specification, the term “imaging slice” may refer to a unit region for obtaining data to generate an image. Also, the term “imaging slab” may refer to a unit region having a plane shape and a thickness. Also, the “imaging slab” may include a plurality of imaging slices.

Furthermore, in the present specification, the term “MRI” may refer to an image of an object obtained by using the nuclear magnetic resonance principle.

Furthermore, in the present specification, the term “pulse sequence” may refer to continuity of signals repeatedly applied by an MRI apparatus. A pulse sequence may include a time parameter of a radio frequency (RF) pulse, for example, repetition time (TR) or echo time (TE).

Furthermore, in the present specification, the term “pulse sequence mimetic diagram” shows an order of events that occur in an MRI apparatus. For example, a pulse sequence mimetic diagram may be a diagram showing an RF pulse, a gradient magnetic field, or an MR signal according to time.

An MRI system is an apparatus for acquiring a sectional image of a part of an object by expressing, in a contrast comparison, a strength of an MR signal with respect to a radio frequency (RF) signal generated in a magnetic field having a specific strength. For example, if an RF signal that resonates only a specific atomic nucleus (for example, a hydrogen atomic nucleus) is irradiated for an instant onto the object that is placed in a strong magnetic field and then such irradiation stops, an MR signal is emitted from the specific atomic nucleus, and thus the MRI system may receive the MR signal and acquire an MR image. The MR signal denotes an RF signal emitted from the object. An intensity of the MR signal may be determined according to a density of a predetermined atom (for example, hydrogen) included in the object, a relaxation time T1, a relaxation time T2, and a blood flow.

MRI systems include characteristics different from those of other imaging apparatuses. Unlike imaging apparatuses such as CT apparatuses that acquire images dependent upon a direction of detection hardware, MRI systems may acquire 2D images or 3D volume images that are oriented toward an optional point. MRI systems do not expose objects and examinees to radiation, unlike CT apparatuses, X-ray apparatuses, position emission tomography (PET) apparatuses, and single photon emission CT (SPECT) apparatuses, may acquire images having high soft tissue contrast, and may acquire neurological images, intravascular images, musculoskeletal images, and oncologic images that are important to precisely describe abnormal tissues.

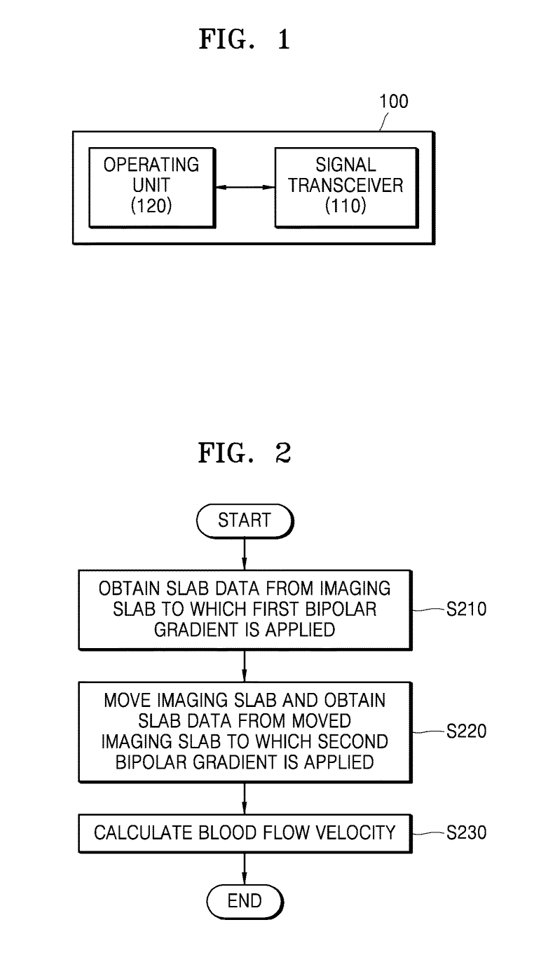

FIG. 1 is a block diagram of a medical imaging apparatus 100 according to an exemplary embodiment.

Referring to FIG. 1, the medical imaging apparatus 100 according to an exemplary embodiment may include a signal transceiver 110 and an operating unit 120 (e.g., operating device).

The signal transceiver 110 may control transmission and reception of a signal generated by the medical imaging apparatus 100.

The signal transceiver 110 may control a gradient magnetic field formed in the medical imaging apparatus 100 according to a signal sequence received from the operating unit 120. According to an exemplary embodiment, the signal transceiver 110 may control a gradient coil so as to apply a first bipolar gradient or a second bipolar gradient to an imaging slab at a pre-set location. The first and second bipolar gradients may sequentially have gradients in opposite polarities (for example, positive and negative poles) having the same magnitude. For example, if a first bipolar gradient sequentially has a positive (+) gradient and a negative (−) gradient having a magnitude, a second bipolar gradient may have a negative gradient and a positive gradient having the same magnitude. Also, the first and second bipolar gradients may be added to a gradient magnetic field in at least one of an x-axis direction, a y-axis direction, and a z-axis direction, and then may be applied to an imaging slab.

Also, the signal transceiver 110 may change a frequency band of an RF pulse so as to continuously obtain slab data of an imaging slab while moving a location of the imaging slab. The continuously obtained slab data may be data to which the first and second bipolar gradients are alternately applied.

Also, the signal transceiver 110 may obtain slice data of imaging slices at a same location on an object, from among the continuously obtained slab data. An imaging slice may denote a unit of data obtained to generate an image, and an imaging slab may include a plurality of imaging slices.

The signal transceiver 110 may obtain data sampled at a sampling rate lower than a reference sampling rate required to reconstruct an image. The sampling rate may be determined by the operating unit 120. The signal transceiver 110 may sample the obtained data based on any one of Cartesian sampling and non-Cartesian sampling such as radial sampling or variable density sampling.

The operating unit 120 may control overall operations of the medical imaging apparatus 100.

The operating unit 120 may generate a pulse sequence for controlling the signal transceiver 110, and transmit the pulse sequence to the signal transceiver 110. A pulse sequence may include any information required to control the signal transceiver 110. For example, a pulse sequence may include information about a magnitude of a signal applied to an object, application duration, and application timing of the pulse signal. According to an exemplary embodiment, the operating unit 120 may transmit, to the signal transceiver 110, a pulse sequence for alternately applying the first bipolar gradient or the second bipolar gradient while moving the imaging slab. For example, if the first bipolar gradient was applied to the imaging slab before being moved, the operating unit 120 may transmit, to the signal transceiver 110, a pulse sequence for applying the second bipolar gradient to the imaging slab that moved.

Also, the operating unit 120 may generate a signal for amplifying a signal obtained by the signal transceiver 110, and transmit the generated signal to the signal transceiver 110. For example, the operating unit 120 may generate various signals to perform processing operations on an obtained MR signal, such as frequency transformation, phase detection, low frequency amplification, and filtering, and transmit the generated various signals to the signal transceiver 110. According to an exemplary embodiment, in order to move the location of the imaging slab, the operating unit 120 may generate a control signal for changing a frequency band of an RF pulse, and transmit the generated control signal to the signal transceiver 110. The operating unit 120 may move the imaging slab in a pre-set direction in at least one slice unit or in a unit smaller than a slice unit.

Also, the operating unit 120 may arrange digital data in a k-space (also referred to as a Fourier domain or a frequency domain) of a memory, and reconstruct an image by performing 2D or 3D Fourier transformation on the digital data.

Also, the operating unit 120 may perform a composing process or a difference calculating process on data obtained by the signal transceiver 110. Examples of a composing process may include an adding process of a pixel and a maximum intensity projection (MIP) process.

According to an exemplary embodiment, the operating unit 120 may generate a first composite image by composing slice data to which the first bipolar gradient is applied from among a plurality of pieces of slice data, and generate a second composite image by composing slice data to which the second bipolar gradient is applied from among the plurality of pieces of slice data. The generated first and second composite images may each be a high resolution image from which an aliasing artifact is removed by composing slice data, and may each be an image wherein the contrast between a blood vessel and a static tissue is averaged.

Also, the operating unit 120 may calculate a blood flow velocity based on the generated first and second composite images. When bipolar gradients having opposite polarities and the same magnitude are respectively applied to two imaging slices at the same location on the object and subtraction is performed on the two imaging slices, a signal generated in a static tissue of the object is offset but a phase shift in proportion to the blood flow velocity may occur in a signal generated in a blood flow of the object. The operating unit 120 may subtract the first and second composite images from each other so as to calculate the blood flow velocity by using a phase difference of signals generated in the static tissue and the blood flow.

Also, the operating unit 120 may generate a third composite image by composing obtained slice data. The operating unit 120 may assign the generated third composite image as an initial input image, and generate a reconstructed image having less artifacts and high contrast by using data of a slice having high contrast as obtained data.

In order to reconstruct an image, the operating unit 120 may apply a method used for dynamic MRI, such as a compressed sensing method, a highly constrained back-projection reconstruction (HYPR) method, or a complex expectation maximization method.

FIG. 1 is only an example for describing an exemplary embodiment, and it is understood that the medical imaging apparatus 100 may further include components other than those shown in FIG. 1, or the components of FIG. 1 may be replaced by equivalent components.

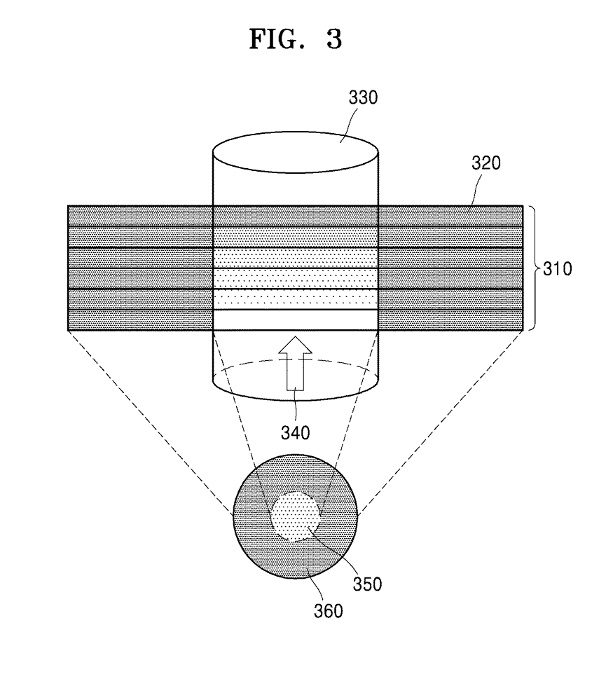

FIG. 2 is a flowchart of a method of measuring a blood flow velocity, according to an exemplary embodiment.

Referring to FIG. 2, in operation S210, the medical imaging apparatus 100 may obtain first slab data from a first imaging slab to which a first bipolar gradient is applied.

The first bipolar gradient may be a gradient magnetic field that sequentially includes positive and negative gradients (or negative and positive gradients) having the same magnitude. According to an exemplary embodiment, the medical imaging apparatus 100 may add a first bipolar gradient to a gradient magnetic field in a z-axis direction and apply the first bipolar gradient to the first imaging slab. Alternatively, the first imaging apparatus 100 may add the first bipolar gradient to each of gradient magnetic fields in x-, y-, and z-axis directions, but exemplary embodiments are not limited thereto.

According to an exemplary embodiment, the medical imaging apparatus 100 may extract first slice data for calculating a blood flow velocity, from the first slab data. Also, the medical imaging apparatus 100 may generate a reconstructed image based on slice data.

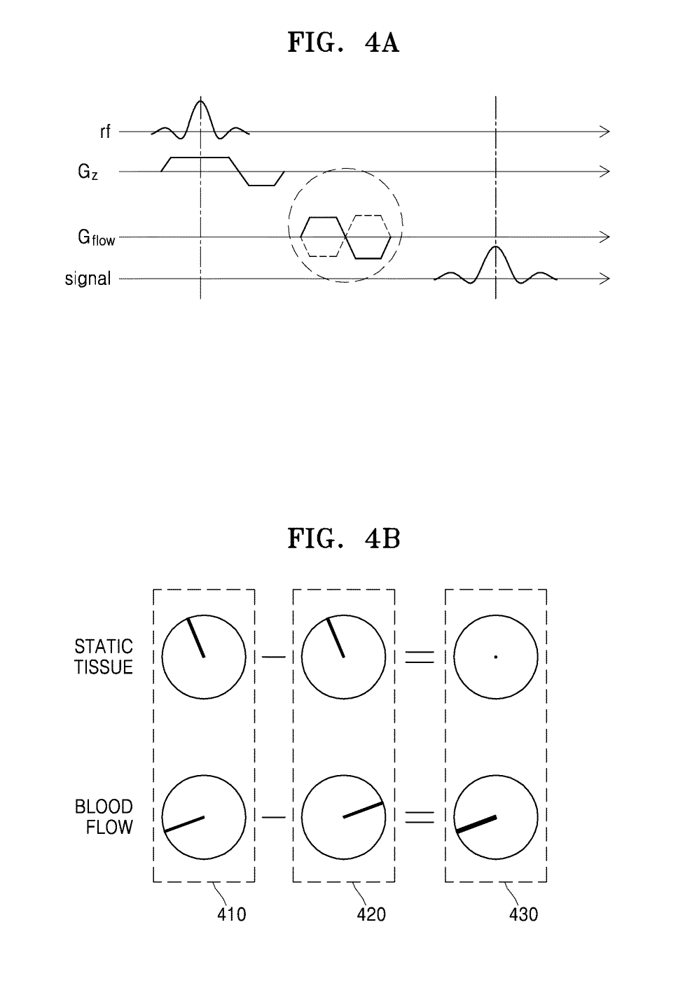

FIG. 3 is a diagram for describing magnetic resonance angiography. The magnetic resonance angiography may refer to a method of obtaining a blood vessel image and additional information by using a difference between a blood vessel and a surrounding tissue, wherein the method is performed by a medical imaging apparatus.

Referring to FIG. 3, an imaging slice 320 may denote a unit region for obtaining data to generate an image. Also, an imaging slab 310 may denote a unit region having a plane shape and a thickness. The imaging slab 310 may include a plurality of the imaging slices 320. For example, one imaging slab 310 may include 20 imaging slices 320.

When an RF pulse is selectively applied to the imaging slab 310 at a certain location on an object, the medical imaging apparatus 100 may receive a reduced signal 360 from a static tissue of the object to which the RF pulse is repeatedly applied. On the other hand, the medical imaging apparatus 100 may receive a signal 350 larger than the reduced signal 360 from a blood flow 340 that newly flows through a blood vessel 330.

According to an exemplary embodiment, the medical imaging apparatus 100 may reconstruct an image by using such magnetic resonance angiography.

Referring back to FIG. 2, in operation S220, the medical imaging apparatus 100 may move the first imaging slab in a pre-set direction, and obtain second slab data from a second imaging slab to which a second bipolar gradient is applied.

The second bipolar gradient may be a gradient magnetic field sequentially including opposite polarities from and having the same magnitude as the first bipolar gradient. For example, if the first bipolar gradient sequentially includes positive and negative gradients having the same magnitude, the second bipolar gradient may sequentially include negative and positive gradients having the same magnitude.

According to an exemplary embodiment, a location of the second imaging slab may be a location moved from a location of the first imaging slab in a certain direction by one slice unit. Alternatively, the location of the second imaging slab may be a location moved from the location of the first imaging slab in a pre-set direction by a plurality of slice units or by a unit smaller than a slice unit. The medical imaging apparatus 100 may change a frequency band of an RF pulse so as to move a location of an imaging slab by at least one slice unit or a unit smaller than a slice unit.

Also, the medical imaging apparatus 100 may extract second slice data for calculating a blood flow velocity from the second slab data. The second slice data may be data obtained from a slice at the same location as the first slice data on the object. Also, the medical imaging apparatus 100 may generate a reconstructed image based on the second slice data.

According to an exemplary embodiment, the medical imaging apparatus 100 may repeat operations S210 and S220 of FIG. 2 a plurality of times to continuously obtain slab data from an imaging slab. In this case, the medical imaging apparatus 100 may alternately apply the first bipolar gradient and the second bipolar gradient as a location of the imaging slab is moved.

In operation S230, the medical imaging apparatus 100 may calculate the blood flow velocity.

According to an exemplary embodiment, the first slice data and the second slice data may be at the same location on the object. Also, the first slice data and the second slice data may be data to which the first bipolar gradient and the second bipolar gradient are respectively applied.

Also, the medical imaging apparatus 100 may generate a reconstructed image based on each of the first slice data and the second slice data. The medical imaging apparatus 100 may calculate the blood flow velocity based on a phase difference between the reconstructed images.

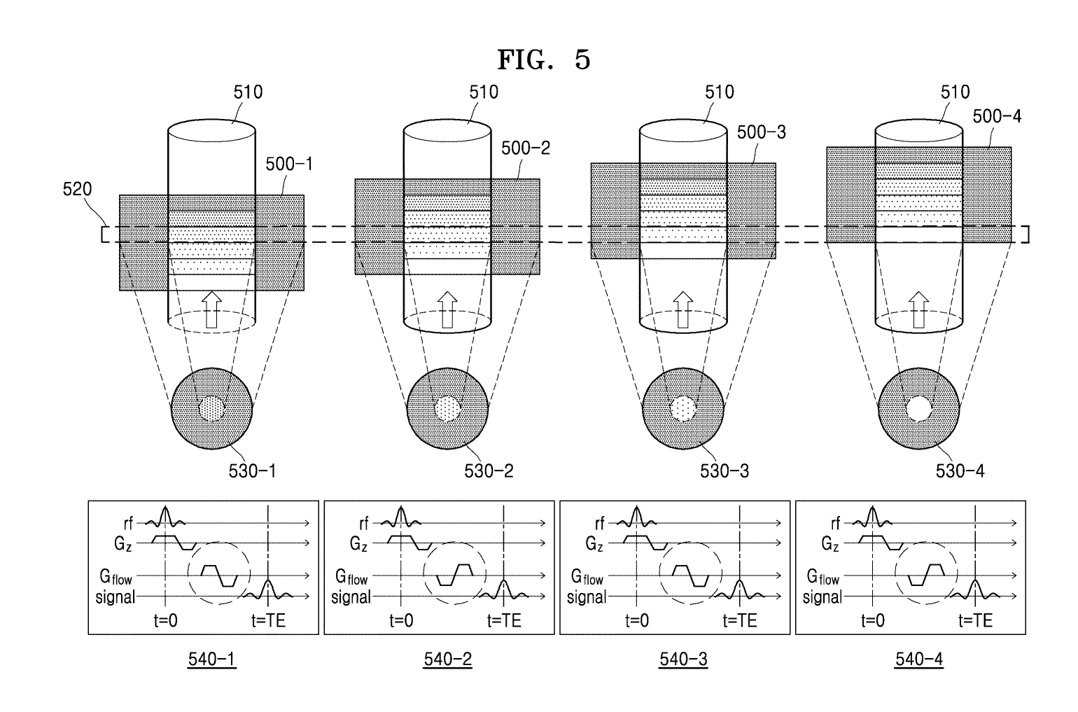

FIGS. 4A and 4B respectively illustrate a pulse sequence mimetic diagram and a diagram for describing phases of a static tissue and blood flow being shifted according to a pulse sequence, according to an exemplary embodiment.

Referring to FIG. 4A, G_{flow} denotes a bipolar gradient. The medical imaging apparatus 100 may apply an RF pulse to an object, and then add G_{flow} indicated by a solid line or by a dashed line to a z-axis gradient magnetic field and apply the z-axis gradient magnetic field to the object. For example, if G_{flow} indicated by the solid line is a first bipolar gradient, G_{flow} indicated by the dashed line may be a second bipolar gradient.

Referring to FIG. 4B, signals obtained from a static tissue and a blood flow of images 410 and 420 reconstructed from slices to which the first bipolar gradient (G_{flow} indicated by the solid line) and the second bipolar gradient (G_{flow} indicated by the dashed line) are respectively applied may have different phase shifts. The medical imaging apparatus 100 may calculate a blood flow velocity by using a phase difference according to the different phase shifts.

According to an exemplary embodiment, the medical imaging apparatus 100 may perform subtraction on images to which a first bipolar gradient and a second bipolar gradient having opposite polarities having the same magnitude are respectively applied. A signal in a static tissue that does not move in a gradient magnetic field may be offset regardless of a magnitude of the gradient magnetic field. On the other hand, a phase of a signal in blood that moves in the gradient magnetic field may shift according to the magnitude of the gradient magnetic field.

According to an exemplary embodiment, the medical imaging apparatus 100 may calculate a blood flow velocity v based on a phase difference Δø according to a signal offset in a static tissue and a phase shift of a blood flow. For example, the medical imaging apparatus 100 may calculate the blood flow velocity v based on equation 1 below.

Δø=γΔm₁ν, where Δm₁=∫₀^{t}G(u)udu−∫₀^{t}G′(u)udu

Here, Δø denotes a phase difference, v denotes a blood flow velocity, and G and G′ respectively denote a first bipolar gradient and a second bipolar gradient applied to a reconstructed image.

According to an exemplary embodiment, the medical imaging apparatus 100 may continuously obtain slab data from a plurality of imaging slabs. In this case, the medical imaging apparatus 100 may extract a plurality of pieces of slice data at the same location on an object, from the obtained slab data. For convenience of description, a number (e.g., starting from 1) is assigned to slice data obtained from imaging slabs according to an order in which the imaging slabs are obtained, wherein slice data to which an odd number is assigned may be data obtained from an imaging slab to which a first bipolar gradient (for example, positive and negative gradients having the same magnitude) is applied, and slice data to which an even number is assigned may be data obtained from an imaging slab to which a second bipolar gradient (for example, negative and positive gradients having the same magnitude) is applied.

According to an exemplary embodiment, the medical imaging apparatus 100 may generate a first composite image reconstructed from a plurality of pieces of slice data in odd numbers, and a second composite image reconstructed from a plurality of pieces of slice data in even numbers. The generated first and second composite images may each be a high resolution image from which an aliasing artifact is removed by composing slice data, and may each be an image wherein contrast between a blood vessel and a static tissue is averaged.

Also, the medical imaging apparatus 100 may calculate a blood flow velocity based on a phase difference between the first and second composite images.

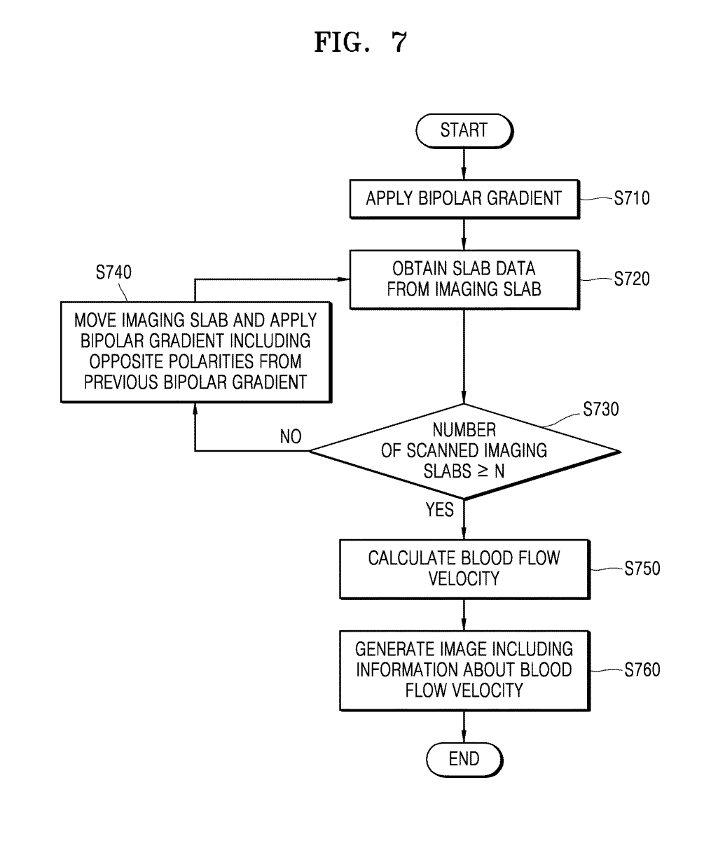

The medical imaging apparatus 100 according to an exemplary embodiment may reconstruct an image having high contrast of blood flow in a blood vessel while including blood flow velocity information, by using a plurality of slice data to which bipolar gradients are applied. A method of reconstructing an image having high contrast will be described later with reference to FIG. 7.

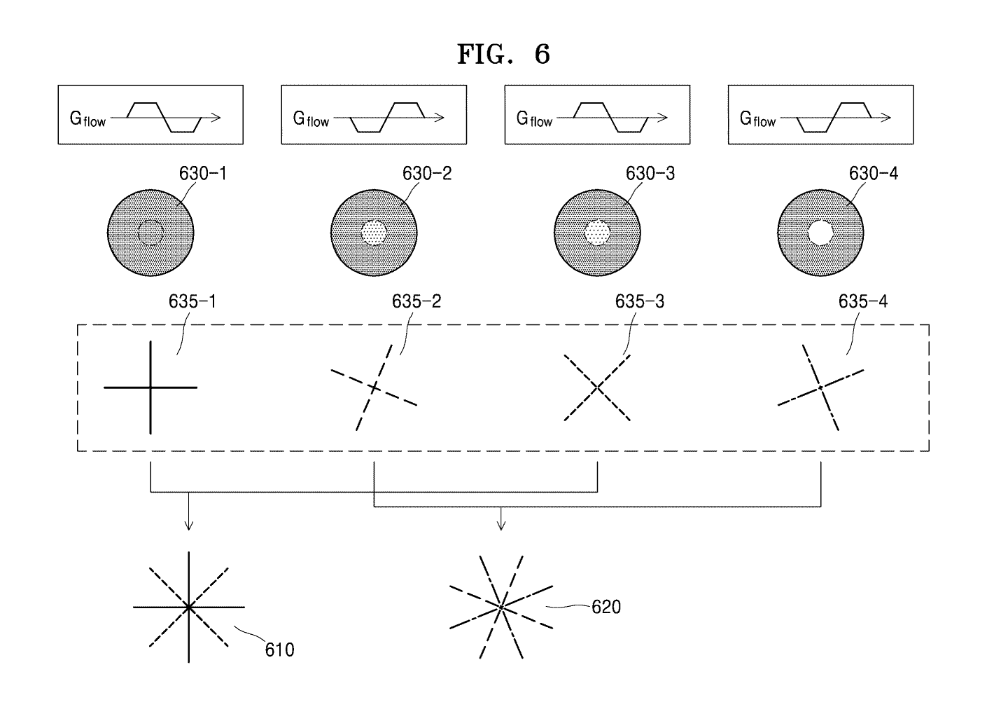

FIGS. 5 and 6 are diagrams for describing a method of measuring a blood flow velocity for the medical imaging apparatus 100, according to an exemplary embodiment.

Referring to FIG. 5, the medical imaging apparatus 100 may obtain slab data from a first imaging slab 500-1 to which a first bipolar gradient 540-1 is applied and a second imaging slab 500-2 to which a second bipolar gradient 540-2 is applied, while moving an image slab from a pre-set location by one slice unit. A first bipolar gradient may sequentially include positive and negative gradients having the same magnitude, and a second bipolar gradient may sequentially include negative and positive gradients having the same magnitude.

Next, the medical imaging apparatus 100 may obtain slab data from a third imaging slab 500-3 to which a first bipolar gradient 540-3 is applied and a fourth imaging slab 500-4 to which a second bipolar gradient 540-4 is applied, while moving an imaging slab.

According to an exemplary embodiment, the medical imaging apparatus 100 may obtain slab data at a reduced sampling rate compared to a reference sampling rate, with respect to each of the imaging slabs 500-1 through 500-4.

According to an exemplary embodiment, the medical imaging apparatus 100 may extract slice data 530-1 through 530-4 at a same location 20 on a blood vessel 510 from the slab data obtained respectively from the imaging slabs 500-1 through 500-4.

In FIG. 5, for convenience of description, first and second bipolar gradients are added to a z-axis gradient magnetic field. However, for 3D imaging, the medical imaging apparatus 100 may add a bipolar gradient to each of x-, y-, and z-axis gradient magnetic fields.

Also, the medical imaging apparatus 100 may calculate a blood flow velocity based on the extracted slice data 530-1 through 530-4. A method of calculating a blood flow velocity, according to an exemplary embodiment, will be described in detail with reference to FIG. 6.

FIG. 6 is a diagram for describing, in detail, a method of calculating a blood flow velocity based on data obtained by the medical imaging apparatus 100, according to an exemplary embodiment.

Referring to FIG. 6, the medical imaging apparatus 100 may obtain slice data via radial sampling. For convenience of description, first, second, third and fourth slice data 630-1, 630-2, 630-3 and 630-4 obtained via radial sampling are respectively conceptually shown as first, second, third and fourth slice data 635-1, 635-2, 635-3 and 635-4.

According to an exemplary embodiment, the medical imaging apparatus 100 may generate a composite image by composing slice data to which bipolar gradients having the same magnitude and the same direction are applied, from among the first, second, third and fourth slice data 635-1, 635-2, 635-3 and 635-4. For example, the medical imaging apparatus 100 may generate first composite data 610 by composing the first slice data 635-1 and the third slice data 635-3 to which a first bipolar gradient is applied, and generate second composite data 620 by composing the second slice data 635-2 and the fourth slice data 635-4 to which a second bipolar gradient is applied.

Also, the medical imaging apparatus 100 may perform subtraction on a first composite image and a second composite image, which are respectively reconstructed from the first composite data 610 and the second composite data 620. By performing the subtraction, a signal of a blood flow may be amplified and a signal of a static tissue may be offset. The medical imaging apparatus 100 may calculate a blood flow velocity based on a phase difference between the offset signal and the amplified signal.

FIG. 7 is a flowchart of a method of reconstructing an image having high contrast of a blood flow in a blood vessel, the image including blood flow velocity information, for the medical imaging apparatus 100, according to an exemplary embodiment.

Referring to FIG. 7, in operation S710, the medical imaging apparatus 100 applies a bipolar gradient sequentially including opposite polarities having the same magnitude to an imaging slab, and in operation S720, the medical imaging apparatus 100 may obtain slab data from the imaging slab to which the bipolar gradient sequentially including opposite polarities having the same magnitude is applied. For example, the bipolar gradient may be a gradient magnetic field sequentially including positive and negative gradients having the same magnitude, or a gradient magnetic field sequentially including negative and positive gradients having the same magnitude.

If it is determined that a number of imaging slabs scanned by the medical imaging apparatus 100 does not exceed a pre-set number N in operation S730, the medical imaging apparatus 100 may move the imaging slab in a pre-set direction and apply a bipolar gradient sequentially including opposite polarities from and having the same magnitude as the previously applied bipolar gradient at operation S740, and may obtain slab data from the moved imaging slab to which the bipolar gradient is applied, in operation S720.

It has been described that the pre-set number N is the number of scanned imaging slabs, but the pre-set number N is not limited thereto and may be a number of scanned imaging slices. Also, the pre-set number N may be automatically set by the medical imaging apparatus 100 or set by a user.

If it is determined that the number of scanned imaging slabs is smaller than or equal to the pre-set number N in operation S730, the medical imaging apparatus 100 may calculate a blood flow velocity based on the obtained slab data in operation S750. Since a method of calculating a blood flow velocity has been described above with reference to operation S230 of FIG. 2, details thereof are not repeated here.

In operation S760, the medical imaging apparatus 100 may reconstruct an image having high contrast of a blood flow in a blood vessel and including information about the calculated blood flow velocity.

According to an exemplary embodiment, the medical imaging apparatus 100 may generate a composite image by composing all pieces of slice data at the same location on a blood vessel from obtained slab data. The composite image may be a high resolution image from which aliasing artifacts are removed and in which contrast between the blood vessel and a static tissue is averaged.

Also, the medical imaging apparatus 100 may assign the generated composite image as an initial input image, and generate a reconstructed image having less artifacts and high contrast by using data of a slice having high contrast as obtained data. Accordingly, all slices of the reconstructed image may have high contrast of the blood flow in the blood vessel. Also, the slices may indicate information about a calculated blood flow velocity. A blood flow in a desired direction may be reconstructed in a bright color. For example, an image including information about artery and vein velocities may be selectively reconstructed from the obtained data.

In order to reconstruct an image, a method used for dynamic MRI, such as a compressed sensing method, a HYPR method, or a complex expectation maximization method, may be applied.

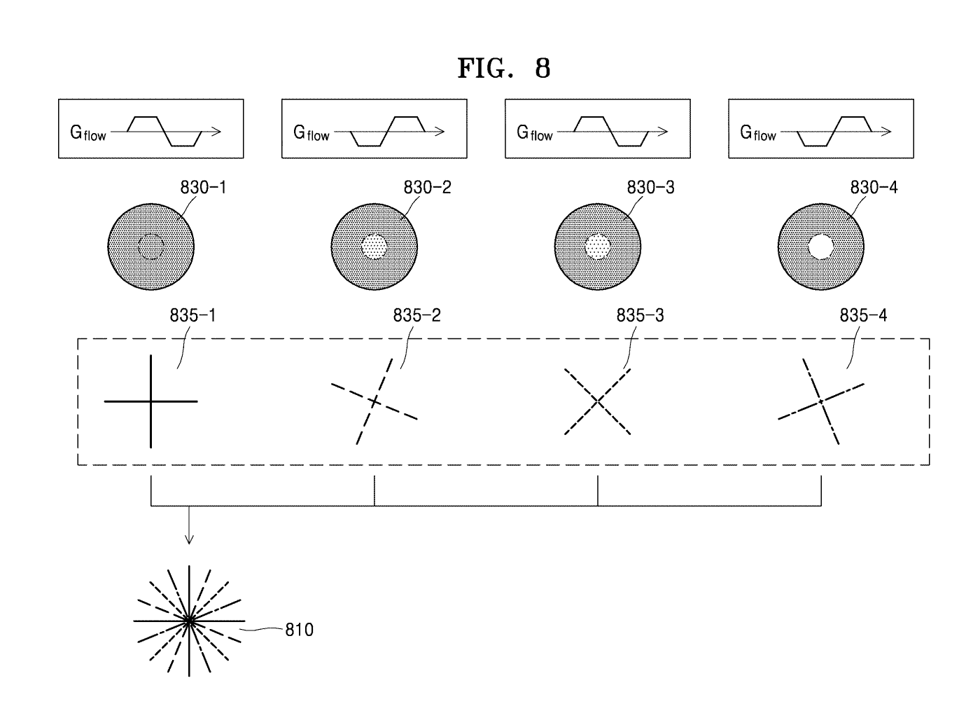

FIG. 8 is a diagram for describing a method of reconstructing an image having high contrast of a blood flow in a blood vessel, the image including blood flow velocity information, for the medical imaging apparatus 100, according to an exemplary embodiment.

For convenience of description, first, second, third and fourth slice data 830-1, 830-2, 830-3 and 830-4 of FIG. 8 are sequentially conceptually shown as slice data 835-1, 835-2, 835-3 and 835-4. Also, it is assumed that a pre-set number is 4.

According to an exemplary embodiment, the medical imaging apparatus 100 may generate a composite image 810 by composing the slice data 835-1 through 835-4. Also, the medical imaging apparatus 100 may assign the composite image 810 as an initial input image, and reconstruct an image having fewer artifacts and high contrast by using the slice data 835-4 of a slice having high contrast as obtained data.

Also, according to an exemplary embodiment, the medical imaging apparatus 100 may assign a weight to each of the slice data 835-1 through 835-4. The medical imaging apparatus 100 may reconstruct an image based on the slice data 835-1 through 835-4 to which the weight is assigned. For example, the medical imaging apparatus 100 may assign a highest weight to the slice data 835-4 having highest contrast, and reconstruct an image having high contrast based on the slice data 835-4 to which the highest weight is assigned.

As such, the medical imaging apparatus 100 may reconstruct an image having high contrast of a blood flow in a blood vessel, the image including blood flow velocity information, based on slab data obtained while moving an imaging slab in a slice unit from a pre-set location.

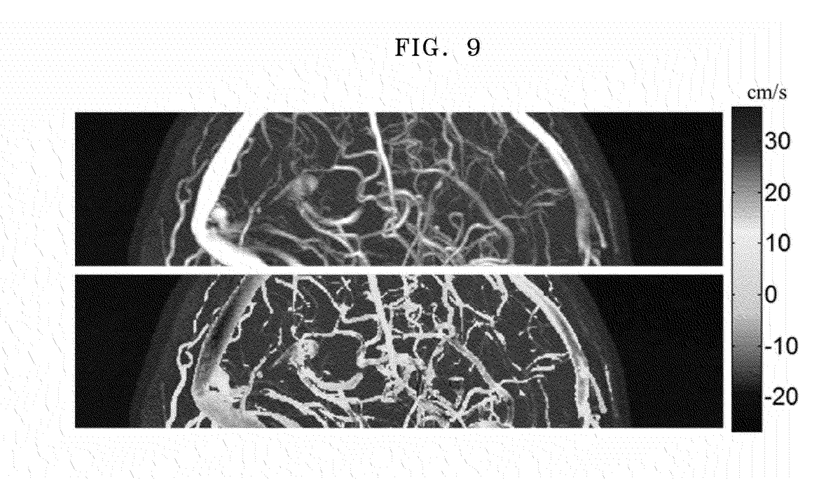

FIG. 9 is a diagram of an example of an image generated by the medical imaging apparatus 100.

FIG. 9 illustrates an image reconstructed based on data obtained from four imaging slabs. The medical imaging apparatus 100 may extract four pieces of slice data at the same location on an object, respectively from the four imaging slabs. The medical imaging apparatus 100 may calculate a blood flow velocity based on the extracted four pieces of slice data, and generate an image having high contrast of a blood flow in a blood vessel.

Referring to FIG. 9, the generated image may include blood flow velocity information. The blood flow velocity information may be displayed in different colors based on a velocity.

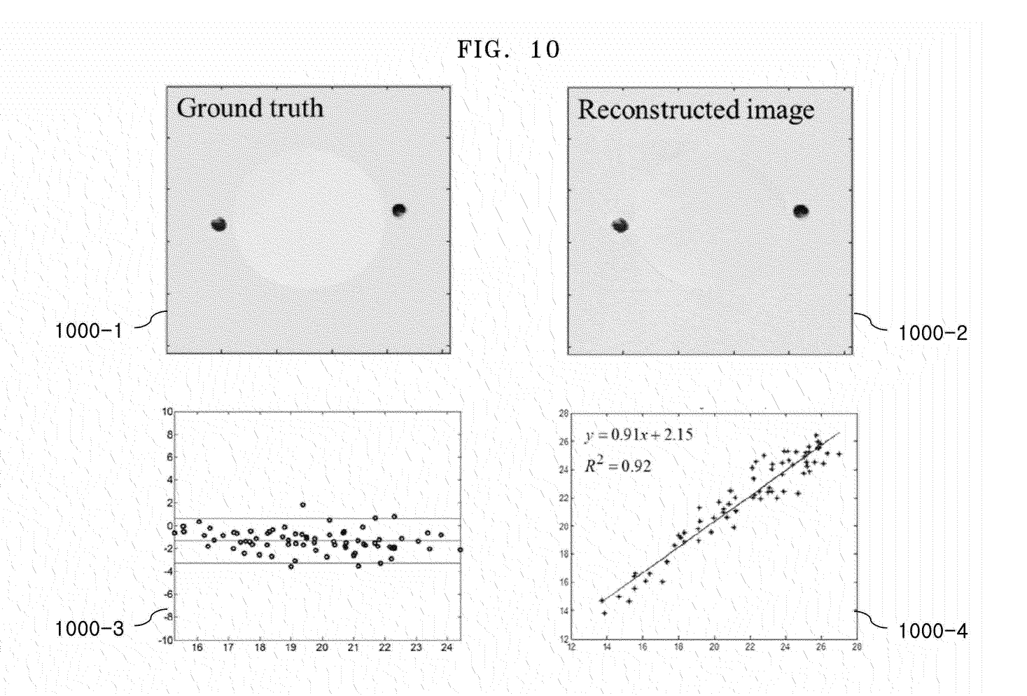

FIG. 10 is a diagram for describing a result of comparing an image reconstructed according to an exemplary embodiment and an image reconstructed via full sampling.

FIG. 10 illustrates a first image 1000-1 obtained via full sampling, a second image 1000-2 obtained according to an exemplary embodiment, and graphs 1000-3 and 1000-4 for comparing a reconstructed degree of the second image 1000-2 with the first image 1000-1, with respect to an object where two water tubes are provided around a phantom bottle.

In the graph 1000-3, X and Y axes respectively denote an average value and a difference value of a blood flow velocity calculated in the first image 1000-1 and a blood flow velocity calculated in the second image 1000-2, based on a Bland-Altman plot. In the graph 1000-3, since plots have values close to 0 in the Y axis, it may be determined that the second image 1000-2 is reconstructed close to the first image 1000-1.

The graph 1000-4 shows the reconstructed degree of the second image 1000-2 based on linear regression. Referring to the graph 1000-4, since a gradient of the graph 1000-4 is close to 1 and a value of R2 is close to 1, it may be determined that the second image 1000-2 is reconstructed similarly to the first image 1000-1.

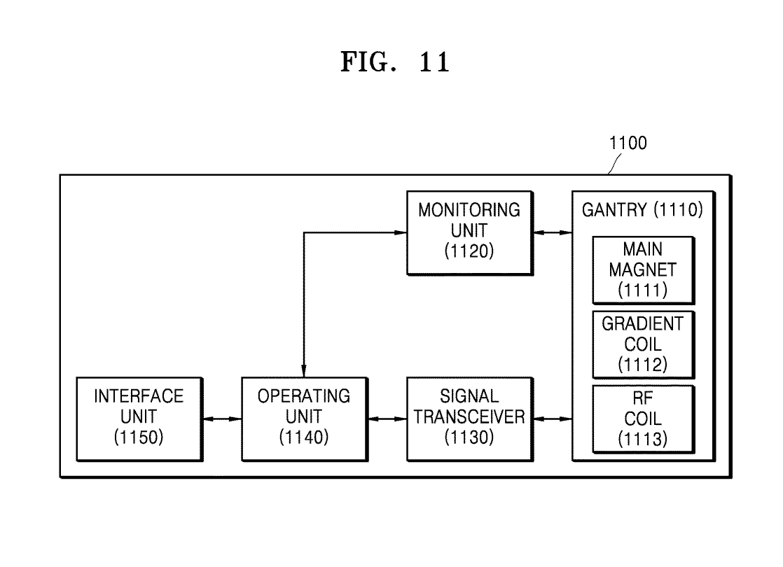

FIG. 11 is a block diagram of a medical imaging apparatus 1100 according to an exemplary embodiment.

Referring to FIG. 11, the medical imaging apparatus 1100 according to an exemplary embodiment may include a signal transceiver 1130 and an operating unit 1140 respectively corresponding to the signal transceiver 110 and the operating unit 120 of FIG. 1, and may further include a gantry 1110, a monitoring unit 1120, and an interface unit 1150 (e.g., interface).

The gantry 1110 may include a main magnet 1111, a gradient coil 1112, and an RF coil 1113, and may block electromagnetic waves generated by the main magnet 1111, the gradient coil 1112, and the RF coil 1113 from being externally emitted. A magnetostatic field and a gradient magnetic field are formed at a bore in the gantry 1110, and an RF signal is irradiated towards an object.

According to an exemplary embodiment, the main magnet 1111, the gradient coil 1112, and the RF coil 1113 may be arranged in a predetermined direction of the gantry 1110. The predetermined direction may be a coaxial cylinder direction. Also, the gantry 1110 may include a table where the object may be disposed.

The main magnet 1111 generates a magnetostatic field or a static magnetic field for aligning a direction of magnetic dipole moments of atomic nuclei in the object in a constant direction. A precise and accurate MR image of the object may be obtained when a magnetic field generated by the main magnet 1111 is strong and uniform.

The gradient coil 1112 includes X, Y, and Z coils for generating gradient magnetic fields in X-, Y-, and Z-axis directions crossing each other at right angles. The gradient coil 1112 may provide location information of each region of the object by differently inducing resonance frequencies according to the regions of the object.

The RF coil 1113 may irradiate an RF signal to the object and receive an MR signal emitted from the object. In detail, the RF coil 1113 may transmit an RF signal at a same frequency as precessional motion to the patient towards atomic nuclei in precessional motion, stop transmitting the RF signal, and then receive an MR signal emitted from the object.

For example, in order to transit an atomic nucleus from a low energy state to a high energy state, the RF coil 1113 may generate and apply an electromagnetic wave signal having an RF corresponding to a type of the atomic nucleus, for example, an RF signal, to the object. When the electromagnetic wave signal generated by the RF coil 1113 is applied to the atomic nucleus, the atomic nucleus may transit from the low energy state to the high energy state. Then, when electromagnetic waves generated by the RF coil 1113 disappear, the atomic nucleus on which the electromagnetic waves were applied transits from the high energy state to the low energy state, thereby emitting electromagnetic waves having a Lamor frequency. In other words, when the applying of the electromagnetic wave signal to the atomic nucleus is stopped, an energy level of the atomic nucleus is changed from a high energy level to a low energy level, and thus the atomic nucleus may emit electromagnetic waves having a Lamor frequency. The RF coil 1113 may receive electromagnetic wave signals from atomic nuclei in the object.

The RF coil 1113 may be realized as a single RF transmitting and receiving coil having both a function of generating electromagnetic waves having a wireless frequency corresponding to a type of an atomic nucleus and a function of receiving electromagnetic waves emitted from an atomic nucleus. Alternatively, the RF coil 1113 may be realized as a transmission RF coil having a function of generating electromagnetic waves having a wireless frequency corresponding to a type of an atomic nucleus, and a reception RF coil having a function of receiving electromagnetic waves emitted from an atomic nucleus.

According to an exemplary embodiment, the RF coil 1113 may be fixed to the gantry 1110 or may be detachable. When the RF coil 1113 is detachable, the RF coil 1113 may be an RF coil for a part of the object, such as a head RF coil, a chest RF coil, a leg RF coil, a neck RF coil, a shoulder RF coil, a wrist RF coil, or an ankle RF coil.

Also, the RF coil 1113 may communicate with an external apparatus via wires and/or wirelessly, and may also perform dual tune communication according to a communication frequency band.

The RF coil 1113 may be a birdcage coil, a surface coil, or a transverse electromagnetic (TEM) coil according to structures. Also, the RF coil 1113 may be a transmission exclusive coil, a reception exclusive coil, or a transmission and reception coil according to methods of transmitting and receiving an RF signal. Also, the RF coil 1113 may be an RF coil in any one of various channels, such as 16 channels, 32 channels, 72 channels, and 144 channels.

According to an exemplary embodiment, the gantry 1110 may further include a display disposed inside and outside the gantry 1110. The medical imaging apparatus 1100 may provide predetermined information to a user or the object through the display disposed outside and inside the gantry 1110.

The monitoring unit 1120 may monitor or control the gantry 1110 or devices mounted on the gantry 1110.

According to an exemplary embodiment, the monitoring unit 1120 may monitor and control a state of a magnetostatic field, a state of a gradient magnetic field, a state of an RF signal, a state of an RF coil, a state of a table, a state of a device measuring body information of an object, a power supply state, a state of a thermal exchanger, and a state of a compressor.

Also, the monitoring unit 1120 monitors a state of the object. For example, the monitoring unit 1120 may include a camera for observing movement or position of the object, a respiration measurer for measuring the respiration of the object, an ECG measurer for measuring ECG of the object, or a temperature measurer for measuring a temperature of the object.

Also, the monitoring unit 1120 may control movement of the table where the object is positioned. For example, during moving imaging of the object, the monitoring unit 1120 may continuously or discontinuously move the table according to the sequence control of the operating unit 1140, and thus the object may be photographed in a field of view (FOV) larger than that of the gantry 1110.

Also, the monitoring unit 1120 may control the display outside and inside the gantry 1110. For example, the monitoring unit 1120 may control a power supply of the display or a screen to be output on the display.

The signal transceiver 1130 may control transmission and reception of a signal generated in the medical imaging apparatus 1100.

The signal transceiver 1130 may control the gradient magnetic field formed inside the gantry 1110, e.g., in the bore, according to a predetermined MR sequence, and control transmission and reception of an RF signal and an MR signal.

Also, the signal transceiver 1130 drives the gradient coil 1112 in the gantry 1110, and may supply a pulse signal for generating a gradient magnetic field to the gradient coil 1112 according to control of the operating unit 1140. By controlling the pulse signal supplied to the gradient coil 1112, the signal transceiver 1130 may compose gradient magnetic fields in X-, Y-, and Z-axis directions.

Also, the signal transceiver 1130 may drive the RF coil 1113. The signal transceiver 1130 may supply an RF pulse in a Lamor frequency to the RF coil 1113, and receive an MR signal received by the RF coil 1113.

Also, the signal transceiver 1130 may adjust transmitting and receiving directions of the RF signal and the MR signal. For example, the RF signal may be irradiated to the object through the RF coil 1113 during a transmission mode, and the MR signal may be received by the object through the RF coil 1113 during a reception mode. The signal transceiver 1130 may adjust the transmitting and receiving directions of the RF signal and the MR signal according to a control signal from the operating unit 1140.

According to an exemplary embodiment, the signal transceiver 1130 may supply a pulse signal to the gradient coil 1112 so as to apply a first bipolar gradient or a second bipolar gradient to an imaging slab at a pre-set location. The first and second bipolar gradients may sequentially include gradients in opposite polarities (positive and negative) having the same magnitude. For example, if the first bipolar gradient sequentially includes positive and negative gradients having a predetermined magnitude, the second bipolar gradient may sequentially include negative and positive gradients having the predetermined magnitude. Also, the first and second bipolar gradients may be added to a gradient magnetic field in at least one of X-, Y-, and Z-axis directions, and then applied to an imaging slab.

Also, according to an exemplary embodiment, the signal transceiver 1130 may continuously obtain slab data of an imaging slab while moving a location of the imaging slab, by changing a frequency band of an RF pulse. The continuously obtained slab data may be data to which the first and second bipolar gradients are alternately applied.

Also, the signal transceiver 1130 may obtain slice data of imaging slices at the same location on an object, from among the continuously obtained slab data. The imaging slice may be a unit for obtaining data to generate an image, and the imaging slab may include a plurality of imaging slices.

The signal transceiver 1130 may obtain data sampled at a sampling rate lower than a reference sampling rate required to reconstruct an image. The sampling rate may be determined by the operating unit 1140. The signal transceiver 1130 may sample obtained data based on any one of radial sampling, variable density sampling, and Cartesian sampling.

The operating unit 1140 may control an overall operation of the medical imaging apparatus 1100.

The operating unit 1140 may control a sequence of signals for controlling the gantry 1110 and the devices mounted on the gantry 1110. Also, the operating unit 1140 may generate a pulse sequence for controlling the signal transceiver 1130, and transmit the generated pulse sequence to the signal transceiver 1130. The pulse sequence may include all information required to control the signal transceiver 1130, for example, may include information about strength, an application time, and an application timing of a pulse signal applied to the gradient coil 1112.

Also, the operating unit 1140 may generate a signal for amplifying a signal obtained by the signal transceiver 1130 from the gantry 1110, and transmit the generated signal to the signal transceiver 1130. For example, the operating unit 1140 may generate various signals for processes, such as frequency transformation of an MR signal, a phase detection, a low frequency amplification, and filtering, and transmit the generated various signals to the signal transceiver 1130.

Also, if required, the operating unit 1140 may perform a composing process or a difference calculating process on data obtained from the signal transceiver 1130. The composing process may include an adding process on a pixel and an MIP process.

According to an exemplary embodiment, the operating unit 1140 may transmit a pulse sequence for alternately applying the first or second bipolar gradient while moving an imaging slab, to the signal transceiver 1130. For example, if the first bipolar gradient was applied to the imaging slab before being moved, the operating unit 1140 may transmit a pulse signal for applying the second bipolar gradient to the imaging slab that is moved, to the signal transceiver 1130.

Also, according to an exemplary embodiment, the operating unit 1140 may generate a control signal for changing a frequency band of an RF pulse so as to move a location of the imaging slab, and transmit the generated control signal to the signal transceiver 1130. The operating unit 1140 may move the imaging slab in a pre-set direction by at least one slice unit or a unit smaller than a slice unit.

Also, the operating unit 1140 may arrange digital data in a k-space (also referred to as a Fourier domain or a frequency domain) of a memory, and reconstruct an image by performing 2D or 3D Fourier transformation on the digital data.

Also, according to an exemplary embodiment, the operating unit 1140 may generate a first composite image by composing slice data to which the first bipolar gradient is applied from among a plurality of pieces of slice data, and generate a second composite image by composing slice data to which the second bipolar gradient is applied. The first and second composite images may each be a high resolution image from which aliasing artifacts are removed by composing slice data, and may each be an image in which a contrast between a blood vessel and a static tissue is averaged.

Also, the operating unit 1140 may calculate a blood flow velocity based on the first and second composite images. The operating unit 1140 may perform subtraction on the first and second composite images so as to calculate a blood flow velocity by using a phase difference of signals generated in the static tissue and the blood flow.

Also, the operating unit 1140 may generate a third composite image by composing slice data. The operating unit 1140 may assign the third composite image as an initial input image, and reconstruct an image having fewer artifacts and high contrast by using slice data having high contrast as obtained data.

Also, the operating unit 1140 may store not only data about the reconstructed image, but also data about the composing process or the difference calculating process, in a memory (not shown) or an external server.

The operating unit 1140 may perform various signal processes in parallel. For example, a plurality of MR signals received through a multi-channel RF coil may be rearranged as image data by performing signal processes on the plurality of MR signals in parallel.

The interface unit 1150 may include an input unit (not shown) and an output unit (not shown) so that a user may communicate with the medical imaging apparatus 1100.

The output unit may output image data reconstructed or rearranged by the operating unit 1140 to the user. Also, the output unit may output information required for the user to manipulate the medical imaging apparatus 1100, such as a user interface (UI), user information, or object information.

According to an exemplary embodiment, the output unit may output image information including information about a blood flow velocity calculated by the operating unit 1140 to the user.

The output unit may include a speaker, a printer, a cathode-ray tube (CRT) display, a liquid crystal display (LCD), a plasma display panel (PDP), an organic light-emitting device (OLED) display, a field emission display (FED), a light-emitting diode (LED) display, a vacuum fluorescent display (VFD), a digital light processing (DLP) display, a PFD display, a 3-dimensional (3D) display, or a transparent display, or any one of various other types of output devices that are well known to one of ordinary skill in the art.

The user may input object information, parameter information, a scan condition, a pulse sequence, or information about image composition or difference calculation by using the input unit. The input unit may include a keyboard, a mouse, a track ball, a voice recognizer, a gesture recognizer, or a touch screen, or may include any one of various other types of input devices that are well known to one of ordinary skill in the art.

The monitoring unit 1120, the signal transceiver 1130, the operating unit 1140, and the interface unit 1150 are exemplarily shown as being separate components in FIG. 11, but it is understood by one of ordinary skill in the art that functions of the monitoring unit 1120, the signal transceiver 1130, the operating unit 1140, and the interface unit 1150 may be performed according to different configurations or components. For example, the signal transceiver 1130 may convert an MR signal into a digital signal, but such a conversion to a digital signal may be performed by the operating unit 1140 or the RF coil 1113.

The gantry 1110, the monitoring unit 1120, the signal transceiver 1130, the operating unit 1140, and the interface unit 1150 may be connected to each other via wires or wirelessly, and when the gantry 1110, the monitoring unit 1120, the signal transceiver 1130, the operating unit 1140, and the interface unit 1150 are connected wirelessly, the medical imaging apparatus 1100 may further include an apparatus (not shown) for synchronizing clocks therebetween. Communication between the gantry 1110, the monitoring unit 1120, the signal transceiver 1130, the operating unit 1140, and the interface unit 1150 may be performed by using a high-speed digital interface, such as low voltage differential signaling (LVDS), asynchronous serial communication, such as a universal asynchronous receiver transmitter (UART), a low-delay network protocol, such as an error synchronous serial communication or controller area network (CAN), or optical communication, or any other communication method that is known to one of ordinary skill in the art.

The exemplary embodiments can be written as computer programs and can be implemented in general-use digital computers that execute the programs using a computer readable recording medium.

Examples of the computer readable recording medium include magnetic storage media (e.g., ROM, floppy disks, hard disks, etc.), optical recording media (e.g., CD-ROMs, or DVDs), and other types of storage media.

While one or more exemplary embodiments have been described with reference to the figures, it will be understood by those of ordinary skill in the art that various changes in form and details may be made therein without departing from the spirit and scope of the exemplary embodiments as defined by the following claims.

What is claimed is:

1. A method of measuring a blood flow velocity of blood flowing in an object, the method comprising:
obtaining first slab data of a first imaging slab to which a first bipolar gradient is applied;
obtaining second slab data of a second imaging slab to which a second bipolar gradient is applied, the second imaging slab being moved to a location different from a location of the first imaging slab; and
calculating the blood flow velocity based on data included in slices of the first slab data and slices of the second slab data, the slices of the first slab data being located at a same location as the slices of the second slab data on the object.

2. The method of claim 1, wherein the first bipolar gradient is a gradient magnetic field that sequentially has positive (+) and negative (−) gradients, and the second bipolar gradient is a gradient magnetic field that has gradients having opposite polarities from and same magnitudes as the first bipolar gradient.

3. The method of claim 2, wherein the first bipolar gradient and the second bipolar gradient are each generated in at least one of an x-axis direction, a y-axis direction, and a z-axis direction.

4. The method of claim 1, wherein the calculating of the blood flow velocity comprises obtaining first slice data and second slice data at a same location on the object, and
wherein the first slice data is extracted from the first slab data and the second slice data is extracted from the second slab data.

5. The method of claim 4, wherein the calculating of the blood flow velocity comprises calculating the blood flow velocity by using a phase difference between images generated based on the first slice data and the second slice data.

6. The method of claim 5, further comprising generating an image comprising information about the calculated blood flow velocity, based on the first slice data and the second slice data.

7. The method of claim 1, wherein the obtaining of the first slab data and the obtaining of the second slab data comprise sampling the first slab data and the second slab data at a sampling rate lower than a reference sampling rate.

8. The method of claim 1, wherein a location of the first imaging slab and a location of the second imaging slab differ from each other by at least one slice unit.

9. The method of claim 1, wherein the obtaining of the first slab data and the obtaining of the second slab data comprise obtaining the first slab data and the second slab data based on radial sampling.

10. A medical imaging apparatus comprising:
a signal transceiver configured to obtain first slab data of a first imaging slab to which a first bipolar gradient is applied, and obtain second slab data of a second imaging slab to which a second bipolar gradient is applied, the second imaging slab being moved to a location different from a location of the first imaging slab; and
an operating device configured to calculate a blood flow velocity of blood flowing in an object based on data included in slices of the first slab data and the second slab data, the slices of the first slab data being located at a same location as the slices of the second slab data on the object.

11. The medical imaging apparatus of claim 10, wherein the first bipolar gradient is a gradient magnetic field that sequentially has positive (+) and negative (−) gradients, and
the second bipolar gradient is a gradient magnetic field that has gradients having opposite polarities from and same magnitudes as the first bipolar gradient.

12. The medical imaging apparatus of claim 11, wherein the first bipolar gradient and the second bipolar gradient are each generated in at least one of an x-axis direction, a y-axis direction, and a z-axis direction.

13. The medical imaging apparatus of claim 10, wherein the signal transceiver is configured to obtain first slice data and second slice data at a same location on the object, and
wherein the first slice data is extracted from the first slab data and the second slice data is extracted from the second slab data.

14. The medical imaging apparatus of claim 13, wherein the operating device is configured to calculate the blood flow velocity by using a phase difference between images generated based on the first slice data and the second slice data.

15. The medical imaging apparatus of claim 14, wherein the operating device is configured to generate an image comprising information about the calculated blood flow velocity, based on the first slice data and the second slice data.

16. The medical imaging apparatus of claim 10, wherein the signal transceiver is configured to obtain the first slab data and the second slab data by sampling the first slab data and the second slab data at a sampling rate lower than a reference sampling rate.

17. The medical imaging apparatus of claim 10, wherein a location of the first imaging slab and a location of the second imaging slab differ from each other by at least one slice unit.

18. The medical imaging apparatus of claim 10, wherein the transceiver is configured to obtain the first slab data and the second slab data based on radial sampling.

19. A non-transitory computer-readable recording medium having recorded thereon a program for executing the method of claim 1.
